(19) United States
(12) Patent Application Publication
(10) Pub. No.: US 2024/0269371 A1
(43) Pub. Date: Aug. 15, 2024

(54) LIQUID DISPENSER FOR NASAL APPLICATIONS

(71) Applicant: Aptar Radolfzell GmbH, Radolfzell (DE)

(72) Inventors: Jürgen GREINER-PERTH, Gottmadingen (DE); Stefan RITSCHE, Eigeltingen (DE)

(21) Appl. No.: 18/568,455

(22) PCT Filed: Jun. 1, 2022
(86) PCT No.: PCT/EP2022/064976

§ 371 Date: Dec. 8, 2023

(30) Foreign Application Priority Data
Jun. 11, 2021 (EP) 21178968.0

(51) Int. Cl. A61M 3/02

(52) U.S. Cl. CPC A61M 3/0254; A61M 3/0279

(57) ABSTRACT

A liquid dispenser having a liquid reservoir for storing liquid, an elongated nasal applicator, a nozzle plate with a plurality of nozzle openings at a distal end of the nasal applicator through which liquid is discharged, and a conveying device conveying liquid from the liquid reservoir to the nozzle openings. The conveying device has a movable pressure element serving to pressurize the liquid for discharge. The conveying device has an actuating handle, displaceable for the purpose of actuation, from a starting position into an actuated end position. The actuating handle and the pressure element are connected via a spring accumulator loaded by displacement of the actuating handle into the end position and which accumulator subsequently moves the pressure element. The actuating force stored in the accumulator subsequently continues to move the pressure element once actuation has already concluded and thus brings about an ongoing discharge.

### FIELD OF APPLICATION AND PRIOR ART

The invention relates to a liquid dispenser for nasal applications. Such a dispenser serves the purpose of introducing pharmaceutical liquids into the nose and the respiratory tract of a patient.

A generic liquid dispenser has a liquid reservoir in which the pharmaceutical liquid is stored prior to discharge as well as an elongated nasal applicator, at the distal end of which at least one discharge opening is provided through which the liquid is discharged.

In order to discharge liquid, there is provided in the case of generic liquid dispensers an actuation which is achieved in particular in the case of output of the liquid in droplet form via a compression of the liquid reservoir and in particular in the case of output of the liquid in an atomized form is performed by an actuating handle which indirectly brings about a pressurization of the liquid.

The present invention relates to liquid dispensers in which the liquid is atomized in the course of the discharge, i.e. is transformed into a mist of very fine liquid droplets. This is performed in the case of most generic dispensers via a vortex chamber arranged upstream of the discharge opening, into which vortex chamber the liquid is tangentially introduced. The liquid is broken up into fine droplets during exit as a result of the rapid rotating movement achieved.

### OBJECT AND SOLUTION

The object of the invention is to make available a liquid dispenser for nasal applications which does not require a vortex chamber and enables a particularly fine atomization of the liquid.

A liquid dispenser according to the invention has a nozzle plate with a plurality of nozzle openings at a distal end of the nasal applicator. The nozzle plate has at least 25 nozzle openings, the average clear cross-sectional surface area of which at the narrowest point is in each case at most 500 μm². However, configurations with significantly more nozzle openings which are preferably configured to be significantly smaller as described below are in particular preferred.

The liquid is output through the nozzle openings in the form of a plurality of fine jets which disintegrate on the other side of the nozzle openings (“Rayleigh breakup”).

As a result of the small nozzle openings which are required to achieve the disintegration of the jet into single droplets, there arises, however, the problem of discharging a sufficient quantity of liquid. The quantity of liquid which should normally be reached during a discharge is normally between 50 μl and 150 μl. Nozzle plates known from other dispensers usually only allow the discharge of a liquid flow of less than 50 μl/second, hence the discharge of the stated quantity of liquid can last three seconds or more. It has, however, been shown in practice that an actuating process of such a duration is unfamiliar to the user and this can lead to premature break off of actuation.

In order to be able to reliably perform the discharge of such a quantity of liquid, it is proposed that the liquid dispenser has a conveying device for conveying the liquid out of the liquid reservoir to the nozzle openings, which conveying device has a movable pressure element which serves in the manner of a piston or another type of displaceable wall to pressurize the liquid.

In order to displace this pressure element, the conveying device has an actuating handle for manual displacement which is displaced in the course of the actuation from a starting position into an actuated end position. It is provided here that the displacement of the actuating handle which takes place is not transmitted directly to the pressure element. Instead, the actuating handle and the pressure element are connected via a spring accumulator which is loaded by displacement of the actuating handle into the end position and which subsequently moves the pressure element once the actuating handle has already reached the end position.

The displacement of the actuating element therefore leads in accordance with its intended use to no or only to a small simultaneous displacement of the pressure element. Instead, the spring element, which can be configured, for example, in the form of an in particular metallic coil spring or leg spring, is tensioned. The movement of the pressure element is essentially driven by this spring element which relaxes again after tensioning once the actuating handle has already reached its end position.

The liquid dispenser is preferably adapted in such a manner that, in the event of intended, sudden displacement of the actuating handle into the end position and thus sudden tensioning of the spring accumulator, the discharge of liquid subsequently brought about by the spring accumulator lasts at least 0.3 seconds, preferably at least 0.5 seconds, in particular preferably at least 1 second. In principle, however, significantly longer periods of time can also be provided, for example, of at least two seconds or even of at least three seconds.

The flow resistance which is generated by the nozzle openings and the liquid pressure brought about by the spring accumulator are above all decisive for the period of discharge.

The stated periods of time of, for example, three seconds are too long in practice in order to expect the patent to continuously exert force on the actuating handle over such a period of time. As a result of the spring accumulator provided according to the invention, the patient can be relieved of this burden since the patient can push the actuating handle directly into the end position and thereby introduce into the system the energy which subsequently brings about the pressurization of the liquid in a continued manner. Were a spring element not present there and were the actuating handle to be connected directly to the pressure element, a direct and rapid displacement of the actuating handle into the end position would not be possible since the discharge of liquid through the nozzle openings cannot discharge liquid quick enough even in the case of significantly elevated pressure.

A liquid dispenser of the described type can be formed in particular in two ways.

In the case of a first variant, the pressure element directly borders the liquid reservoir. The pressure generated by the pressure element therefore leads to pressurization of the entire liquid. It can be provided in particular in the case of such a variant that the liquid in the liquid reservoir is discharged in the course of a single actuation or in the course of two actuations. Corresponding dispensers are also referred to as unit dose dispensers or bidose dispensers. In the case of such a configuration, the pressure element is preferably part of a component which is configured in the manner of a cylinder closed on one side which is displaced as a whole with respect to a piston which is stationary in relation to the nasal applicator.

In the case of a second variant, the pressure element borders a pump chamber, in particular in the manner of a piston which is displaceable in the pump chamber. This pump chamber is part of a pump device. It is connected via an input channel with an inlet valve to the liquid reservoir and via an output channel with an outlet valve to the nozzle openings. The displacement of the pressure element driven indirectly via the actuating handle and directly via the spring accumulator corresponding in the case of this variant does not lead to a pressurization of the liquid in the liquid reservoir, rather only to a pressurization of the liquid in the pump chamber and downstream thereof.

In the simplest case, it can be provided in the case of a liquid dispenser of the described type that the patient pushes the actuating handle into the end position and keeps pushing it until the spring accumulator has relaxed and the discharge ends.

Since this, however, involves the risk of incorrect operation by premature termination of the action of force, it is regarded as preferred if an automatically latching latching device is provided on a housing portion which is stationary with respect to the nasal applicator, by means of which the actuating handle is secured in its end position. Such a latching device comprises in particular preferably an elastically deflectable latching element with a slant which is deflected by the actuating handle or a sub-element moved together with the actuating handle during actuation and latches with the actuating handle or the stated sub-element under the effect of the elastic deflection when the end position is reached. Instead of a housing portion, the deflectable latching device can also be provided on the actuating handle or another sub-element moved with it.

The latching device brings about that the patient can immediately end the action of force after actuation of the actuating handle since the end position is now maintained by the latching device. The latching device is furthermore advantageous since it makes it clear to the patient that he or she has reached the end position by means of an audible or perceptible latching in.

In particular in the case of a dispenser which is provided for single use, this latching device is preferably provided as an undetachable latching device. This means that the patient does not have any possibility in accordance with the intended use to release the latching again. In particular, the latching device can be arranged at a location of the dispenser which cannot be accessed by the patient, in particular protected by surrounding walls of the dispenser.

Alternatively, the latching device can be formed as a latching device which is detachable in accordance with the intended use. This is expedient in particular in the case of the configuration described above of the liquid dispenser with a pump device since here the actuating handle must be able to return to its starting position for a further actuation.

In the case of a detachable latching device, a separate release button is preferably provided through which the latching is released. The elastically deflectable latching element described above can in particular be displaced by this release button so that it releases the actuating handle. Under the effect of the spring accumulator and/or a separate piston spring of the pump, the actuating handle is then pushed back into the starting position.

Two designs of a dispenser according to the invention are preferred. In the case of the first of these designs, it is provided that the actuating handle is translationally displaceable in the direction of a main direction of extent of the nasal applicator and/or a main direction of discharge, wherein the main direction of extent and the main direction of discharge are preferably identical. In particular, it is advantageous if the actuating handle is provided on the dispenser opposite the nasal applicator and is pushed upward in the direction of the nasal applicator.

For ease of operation, at least one counterforce surface for manual support is preferably provided in a stationary manner with respect to the nasal applicator. In particular, this can involve two counterforce surfaces which are provided on both sides of the nasal applicator. In accordance with the intended use, the index finger and the middle finger are placed on these two counterforce surfaces, while the thumb is placed on the actuating handle and pushes it in the direction of the counterforce surfaces in the course of the actuation.

The second, particularly preferred design provides that the liquid dispenser has an elongated housing oriented in the direction of a main direction of extent, at the distal end of which the nozzle openings are provided and at the opposite end of which the liquid reservoir is preferably provided. In the case of this design, the actuating handle is arranged to the side of the housing and thus eccentrically with respect to a central axis. The actuation of the actuating handle is performed in the direction of the central axis, wherein both a pivotably movable actuation and a translational actuation can be provided. Such a dispenser with a laterally attached actuating handle is also referred to as a side-actuation dispenser.

The stated second design is expedient in particular in the case of a dispenser with a pump device. It is in particular preferably provided that the pressure element is displaceable orthogonally to the main axis of extent of the dispenser, the pump device is therefore installed transversely into the housing. Configurations are nevertheless also conceivable in which further transmission elements are provided between the actuating handle and the pressure element, which transmission elements serve the purpose of a conversion of the force or the direction of force so that a pump device oriented in the main direction of extent is possible.

The problem described above that the fine nozzle openings make it necessary to have a long-lasting discharge can also be overcome by a liquid dispenser which is formed in the manner described above as a liquid dispenser for nasal applications and has a liquid reservoir as well as a nasal applicator with a nozzle plate, in the case of which, however, the additional peculiarity arises that the nozzle plate is designed for a comparatively large liquid flow.

According to the invention, it is provided in this case that all the nozzle openings are designed in such a manner that a total liquid flow of at least 100 μl/second through the nozzle openings is achieved if liquid with the properties of water in an antechamber arranged upstream of the nozzle plate under a pressure of 5 bar bears against the nozzle plate.

When using such a nozzle plate, the discharge quantity of 50 μl to 150 μl, which is normally provided for each discharge process in the case of discharge of pharmaceutical liquids by means of nasal applicators, can be discharged in 1.5 seconds or less.

Although in the manner described above a liquid dispenser which has a spring accumulator and in particular also a latching device of the described type is advantageous, when using such a nozzle plate designed for large liquid flow and thus in the case of a discharge period of preferably 1.5 seconds or less, the spring element and also the latching device can be dispensed with and instead it can be provided that the patient displaces the pressure element directly via the actuating handle and thus brings about the pressurization of the liquid. Nevertheless, the use of a dispenser of the type described above with a spring accumulator is preferred.

Configurations of the nozzle plate which are designed for a total liquid flow between 100 μl/second and 750 μl/second at 5 bar liquid pressure, in particular preferably for a total liquid flow between 200 μl/second and 500 μl/second, are particularly preferred.

There are various possibilities to achieve such very large liquid flows. One configuration has been shown to be particularly preferred in which between 400 and 2000 nozzle openings are provided. The nozzle openings have in each case a minimum clear cross-sectional surface area between 10 μm² and 20 μm², preferably between 10 μm² and 15 μm². The nozzle openings are preferably formed to be round and have a corresponding minimum diameter, in particular between 1.5 μm and 3 μm.

The production of such small nozzle openings is technically complex, hence this also increases the price of the liquid dispenser.

It has, however, been shown that good results can also be achieved with a nozzle plate with 200 to 1000 nozzle openings. In this case, the nozzle openings are configured to be slightly larger and have a minimum clear cross-sectional surface area between 20 μm and 50 μm², preferably between 25 μm² and 35 μm². In the case of round nozzle openings, their diameter lies in the range between 2.5 μm and 4 μm.

Production can be yet further simplified in that an even smaller number of nozzle openings are provided, namely between 50 and 250 nozzle openings which have in each case a minimum clear cross-sectional surface area between 50 μm² and 100 μm², preferably between 70 μm² and 85 μm². In the case of round nozzle openings, their diameter lies in the range between 4 μm and 6 μm.

These comparatively large nozzle openings are not ideal in terms of atomization. They nevertheless make it possible to achieve the stated high total liquid flow of at least 100 μl/second with low production costs. This design is therefore expedient for low-cost single-use dispensers.

The above-mentioned cross-sectional surface areas are preferably identical for all nozzle openings. Insofar as the nozzle openings are configured differently in terms of their clear cross-sectional surface area, it is regarded as preferred if at least 80% of the nozzle openings have the minimum cross-sectional surface areas stated in each case.

The production of the nozzle plate can be performed in various ways. One particularly preferred configuration provides that the nozzle plates are formed as metallic nozzle plates and produced by electroforming. Here, metal, for example, copper or nickel, are electrolytically separated from an aqueous salt bath so that the nozzle plate is built up in an additive manner.

An alternative technique provides that the nozzle openings are incorporated by laser into a blank. The blank of the nozzle plate can be produced as a thin metallic nozzle plate, ceramic nozzle plate, nozzle plate composed of glass or another mineral material or nozzle plate composed of plastic, into which the nozzle openings have subsequently been incorporated via a laser beam or preferably many laser beams.

A third possibility lies in producing the nozzle plate from plastic, in particular from polypropylene, by injection molding and directly keeping the nozzle openings free of material through the shaping of the cavity used.

As a result of the high number of nozzle openings, it is particularly important to prevent the exiting jets of liquid from coming into contact with one another and thereby preventing a fine atomization. The nozzle openings are therefore preferably provided in a diverging orientation, wherein external nozzle openings are at a greater angle with respect to a central discharge direction than the internal nozzle openings. When producing the nozzle openings by laser or by electroforming, this can be realized directly in the flat nozzle plate. It can in particular, however, also be achieved in that the nozzle plate in the liquid dispenser in an arched shape is used. Such an arching can be forcibly brought about, for example, by an annular plastic carrier into which the nozzle plate is inserted.

### BRIEF DESCRIPTION OF THE DRAWINGS

Further advantages and aspects of the invention will become apparent from the claims and the following description of preferred exemplary embodiments of the invention which are explained below on the basis of the figures.

FIG. 1 shows a first exemplary embodiment of a dispenser according to the invention.

FIGS. 2 to 4 show the nozzle unit of the dispenser of FIG. 1 as well as its nozzle plate in a sectional view and in a plan view.

FIGS. 5A to 5D show the procedure of actuating the dispenser of FIG. 1.

FIGS. 6A to 6D show a second exemplary embodiment of a dispenser according to the invention as well as the procedure for actuating this dispenser.

### DETAILED DESCRIPTION OF THE EXEMPLARY EMBODIMENTS

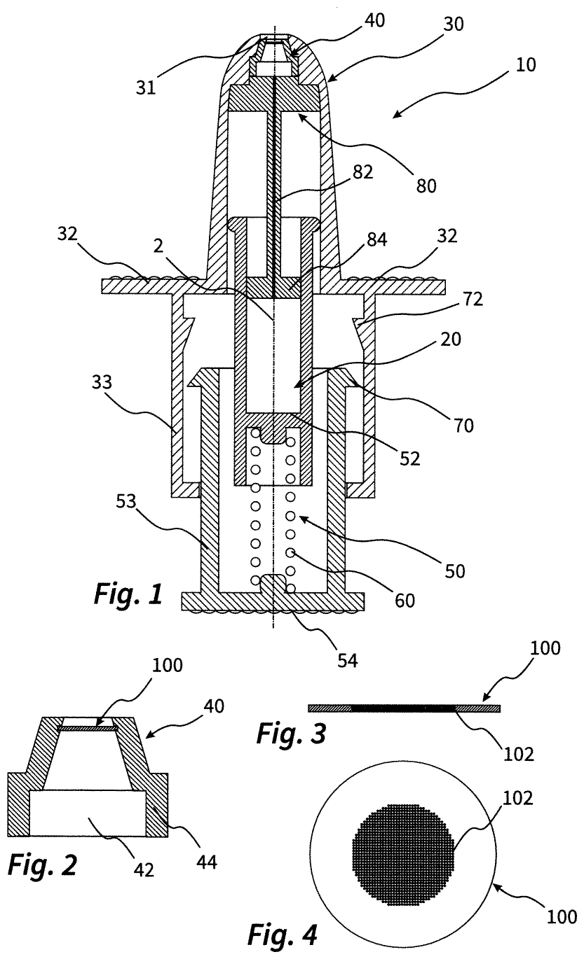

FIG. 1 shows a liquid dispenser 10 according to the invention according to a first exemplary embodiment.

The liquid dispenser 10 is provided as a liquid dispenser for nasal applications and has for this purpose a nasal applicator 30, at the distal end of which an opening 31 is provided, through which liquid can be discharged. The nasal applicator 30 is formed on the outside by a housing component which has still further sub-components which are explained below.

For the purpose of liquid discharge, a conveying device 50 is provided which has a sleeve-shaped actuating unit 53 which is pushed from below into a guide shaft 33 which is stationary in relation to the nasal applicator. The lower side of the actuating unit 53 is formed by an actuating handle 54. Counterforce surfaces 32 thereto are provided projecting on both sides from the nasal applicator 30.

A nozzle unit 40 which is represented in greater detail in FIG. 2 is provided on the opening 31 at the distal end of the nasal applicator 30. The nozzle unit 40 has a plastic sleeve 44 into which a nozzle plate 100 is inserted. An antechamber 42 in which pressurized liquid collects is provided upstream of the nozzle plate 100 in order to then be discharged through nozzle openings 102 of the nozzle plate 100.

Once again with reference to FIG. 1, an inner component 80 is inserted into the nasal applicator 30 upstream of the nozzle unit 40, through which inner component 80 a discharge channel 82 penetrates. The opposite end of the inner component 80 is configured in the manner of a piston 84 which bears with its outside in a sliding and sealing manner against the inside of a cylindrical liquid reservoir 20, the base of which is formed by a pressure element 52.

The liquid reservoir 20 is axially displaceable with respect to the inner component 80 and the nasal applicator, wherein the actuating unit 53 already described is provided to displace the liquid reservoir 20 and the pressure element 52.

However, the actuating unit 53 on one hand and the liquid reservoir 20 with pressure element 52 on the other hand are not connected directly to one another, rather are coupled via a spring accumulator 60 in the form of a coil spring.

FIGS. 3 and 4 show the nozzle plate 100 through which the discharge of the liquid is performed. The nozzle plate can be composed, for example, of metal or ceramic. Around 1200 nozzle openings 102 are provided which penetrate through the nozzle plate and which have in each case a minimum clear cross-sectional surface area of approximately 10 μm². In the case of a liquid pressure of 5 bar which prevails upstream, the nozzle openings jointly ensure a liquid flow of more than 100μ/second.

The nozzle openings 102 can be oriented parallel to one another. However, it is preferably provided that the nozzle openings 102 are oriented in a diverging manner, wherein the external nozzle openings 102 diverge to a greater extent from the main direction of extent 2 than the internal nozzle openings 102. As a result of this, it is ensured that the disintegration of the exiting jets of liquid into single droplets takes place in the desired manner.

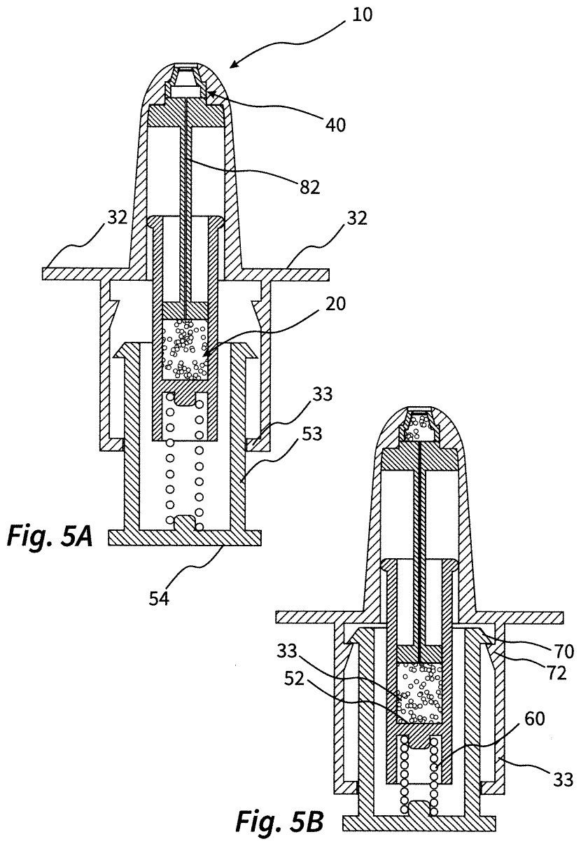

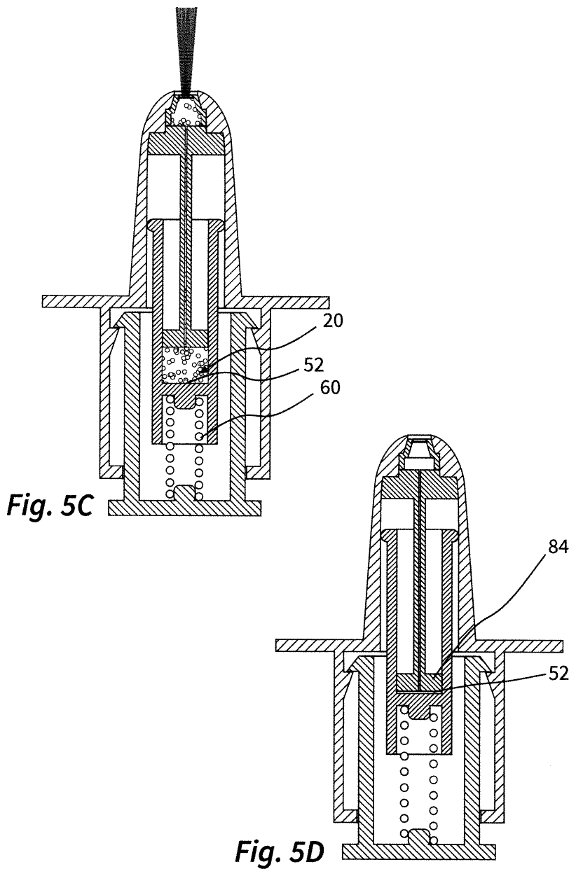

The handling of the liquid dispenser of FIG. 1 is explained on the basis of FIGS. 5A to 5D.

FIG. 5A shows an initial state of the liquid dispenser 10 which corresponds to the state of FIG. 1. The liquid dispenser 20 is filled with a pharmaceutical liquid which can travel through the discharge channel 82 to the nozzle unit 40. It can, however, be provided in a manner not represented in the initial state that the discharge channel 82 is still closed off, for example by a membrane which can be destroyed according to the intended use under the effect of liquid pressure. In the initial state of FIG. 5A, it is preferably provided in a manner not represented in greater detail that the actuating unit 53 and the lower end of the shaft 33 are latched with one another or connected to one another by plastic bridges so that an unintentional pushing in of the actuating unit 53 is prevented and an intentional pushing in involves a tactile pressure point.

Starting from the state of FIG. 5A, the patient grips the liquid dispenser 10 in such a manner that his/her index finger and his/her middle finger are placed on the counterforce surfaces 32, while he/she places his/her thumb on the actuating handle 54 of the actuating unit 53. Proceeding from this, the actuating unit 53 is acted upon by force upward by means of the thumb, wherein the above-mentioned latching is released or the above-mentioned plastic bridges are broken.

The actuating force is also transmitted directly via the spring accumulator 60 to the pressure element 52 and the liquid reservoir 20. However, a majority of the energy introduced is initially absorbed by compression and thus tensioning of the spring accumulator 60. An immediate discharge of the liquid out of the liquid reservoir 20 only takes place to a very restricted extent since the nozzle plate 100 only allows the stated low level of liquid flow.

If the actuating unit 53 is fully pushed in, it latches by means of latching elements 70, 72 with an inner side of the shaft 33. This state is represented in FIG. 5B. As a result of the latching, the actuating unit 53 remains in the pushed-in state. The spring accumulator 60 which is under significant tension furthermore pushes on the pressure element 52 and the liquid reservoir 20 so that the volume of the liquid reservoir 20 is reduced and liquid is discharged over the next, for example, 1 to 2 seconds, as illustrated in FIG. 5C.

It is only if the liquid reservoir 20 has been displaced so far upward that the piston-like end 84 of the inner component 80 bears against the base of the liquid reservoir that the discharge of liquid ends. This is illustrated in FIG. 5D.

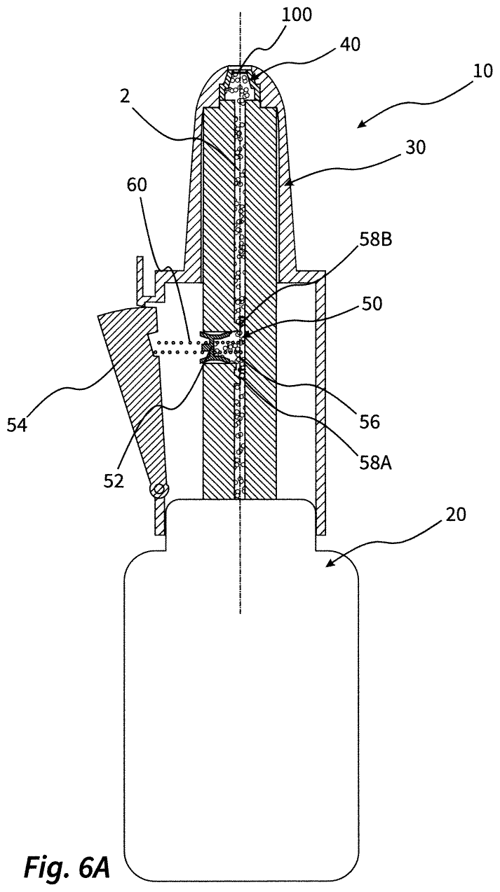

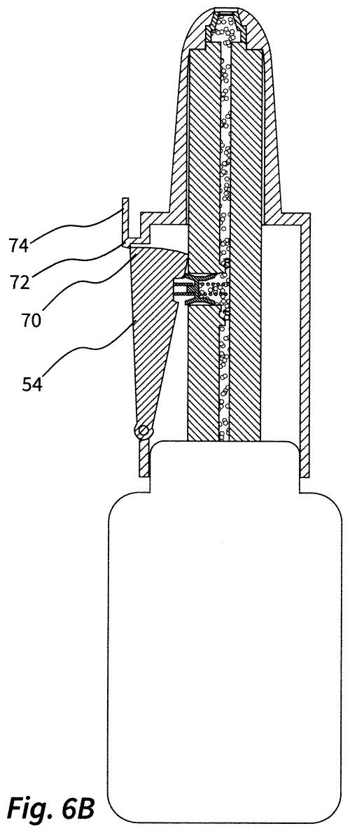

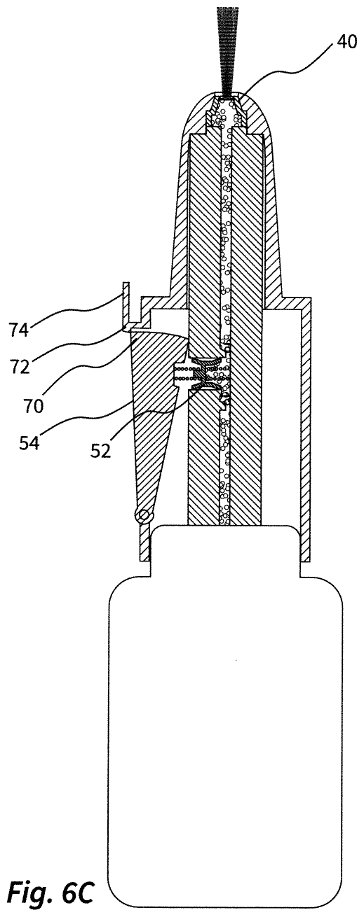

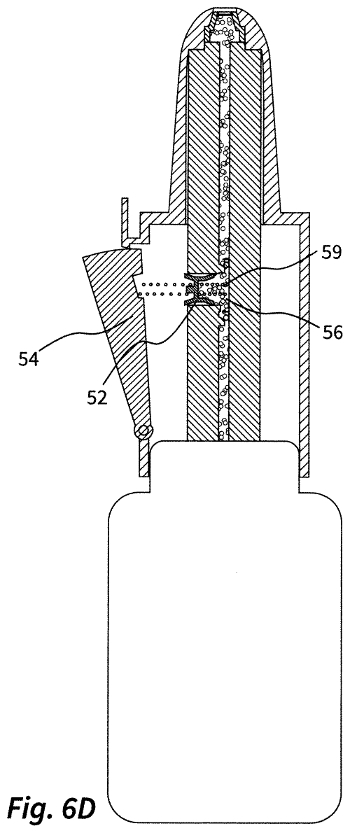

FIGS. 6A to 6D show a second liquid dispenser 10 according to the invention which is likewise provided as a liquid dispenser for the nasal application of pharmaceutical liquids. It therefore likewise has a nasal applicator 30, at the distal end of which a nozzle unit 40 corresponding to FIGS. 2 to 4 is provided.

In contrast to the liquid dispenser of FIG. 1, the liquid dispenser 10 of FIGS. 6A to 6D is, however, formed as a pump dispenser. Its conveying device 50 therefore comprises a pump apparatus with a pump chamber 56 which is connected via an inlet channel to the liquid reservoir 20 and which is connected via an outlet channel to the nozzle unit 40. The pump chamber 56 is bordered by a pressure element 52 the form of a piston. A valve 58A, 58B which opens and closes in a pressure-dependent manner is provided in each case in the inlet channel and in the outlet channel so that, in the case of an increase in size of the pump chamber, liquid is sucked out of the liquid reservoir 20 by displacement of the pressure element 52 to the left and liquid is conveyed out of the pump chamber 56 in the direction of the nozzle unit 40 by displacement of the pressure element 52 to the right.

In order to actuate the liquid dispenser of FIGS. 6A to 6D, a pivotably movable actuating handle 54 is provided on a lateral surface of the housing.

It is in turn provided that a spring accumulator 60 is present between the actuating handle 54 and the pressure element 52.

Proceeding from the initial state of FIG. 6A, in the case of actuation, the actuating handle 54 is initially pushed in so that it assumes the state of FIG. 6B. In this state, it is held by latching elements 70, 72 so that it initially cannot return to the initial state of FIG. 6A. As a result of the displacement of the actuating handle 54 into its end position, the spring accumulator 60 has been fully compressed so that it, starting from the state of FIG. 6B, displaces the pressure element 52 which acts as a piston in the direction of the pump chamber 56 and reduces its volume. The liquid present in the pump chamber is conveyed through the discharge channel to the nozzle unit 40 and is discharged through the nozzle openings 102, as represented in FIG. 6C.

As soon as the discharge ends, the patent pushes in accordance with the intended use on the release button 74, by means of which he/she detaches the latching elements 70, 72 from one another, by means of which the actuating handle 54 was held in its end position. As is represented in FIG. 6D, the actuating handle 54 jumps as a result of this release back into its starting position and a return spring 59 of the pump device pushes the pressure element 52 which acts as a piston to the left. The pump chamber 56 increases in size again and liquid is sucked out of the liquid reservoir 20.

The initial state of FIG. 6A is thus produced again and renewed discharge of liquid can be performed.

1. A liquid dispenser for nasal applications, the liquid dispenser comprising:
a liquid reservoir storing liquid prior to discharge;
an elongated nasal applicator;
a nozzle plate having a plurality of nozzle openings provided at a distal end of the nasal applicator; and
a conveying device for conveying the liquid from the liquid reservoir to the nozzle openings, the conveying device comprising:
a spring accumulator;
a movable pressure element serving to pressurize the liquid for discharge; and
an actuating handle for manual displacement from a starting position into an actuated end position,
the actuating handle and the pressure element being connected by the spring accumulator, the spring accumulator being loaded by displacement of the actuating handle into the end position and the spring accumulator subsequently moving the pressure element once the actuating handle has already reached the end position.

2. The liquid dispenser as claimed in claim 1, wherein
the pressure element directly borders the liquid reservoir.

3. The liquid dispenser as claimed in claim 1, wherein
the conveying device has a pump device with a pump chamber, the pump chamber having an inlet channel with an inlet valve and an outlet valve, wherein the inlet channel connects the pump chamber to the liquid reservoir and the outlet channel connects the pump chamber to the nozzle openings, and
the pressure element forms a pump piston by which the pump chamber can be reduced in size or increased in size.

4. The liquid dispenser as claimed in claim 1, further comprising
an automatically latching device, the latching device securing the actuating handle in the end position.

5. The liquid dispenser as claimed in claim 1, wherein
the actuating handle is translationally displaceable in a direction corresponding to a main direction of extent of the nasal applicator.

6. The liquid dispenser as claimed in claim 1, further comprising
an elongated housing oriented in a direction of a main direction of extent, and
the actuating handle is arranged eccentrically with respect to a central axis of the housing and is, in the case of actuation, displaced at least also radially in the direction of the central axis.

7. The liquid dispenser as claimed in claim 1, wherein:
the spring accumulator is formed in the manner of a coil spring or in the manner of a leg spring; and/or
the liquid reservoir is filled with a pharmaceutical liquid; and/or
the liquid dispenser is configured such that, in the event of displacement of the actuating handle into the end position, the discharge of liquid subsequently brought about by the spring accumulator lasts at least 0.3 seconds.

8. A liquid dispenser for nasal applications, the liquid dispenser comprising:
a liquid reservoir storing liquid prior to discharge;
an elongated nasal applicator; and
a nozzle plate having a plurality of nozzle openings provided at a distal end of the nasal applicator, wherein
all of the nozzle openings are configured such that a total liquid flow of at least 100 μl/second through the nozzle openings is achieved if liquid with the properties of water in an antechamber arranged upstream of the nozzle plate under a pressure of 5 bar bears against the nozzle plate.

9. The liquid dispenser as claimed in claim 8,
wherein the total liquid flow through the nozzle openings is between 100 μl/second and 750 μl/second.

10. The liquid dispenser as claimed in claim 8, wherein the plurality of nozzle openings comprises
between 400 and 2000 nozzle openings, each of the nozzle openings comprising a minimum free cross-sectional surface area between 10 μm² and 20 μm².

11. The liquid dispenser as claimed in claim 8, wherein the plurality of nozzle openings comprises
between 200 and 1000 nozzle openings, each of the nozzle openings comprising a minimum free cross-sectional surface area between 20 μm² and 50 μm².

12. The liquid dispenser as claimed in claim 8, wherein the plurality of nozzle openings comprises
between 50 and 250 nozzle openings, each of the nozzle openings having a minimum free cross-sectional surface area between 50 μm² and 100 μm².

13. The liquid dispenser as claimed claim 1, wherein:
the nozzle plate is formed as a metallic nozzle plate produced by electroforming; or
the nozzle plate is formed as a metallic nozzle plate, as a ceramic nozzle plate, as a nozzle plate composed of glass or another mineral material or as a nozzle plate composed of plastic.

14. The liquid dispenser as claimed in claim 1, wherein
the nozzle openings have a diverging orientation.

15. The liquid dispenser as claimed in claim 4, wherein the latching device is formed as a detachable latching device and has a release button for releasing the latching device.

16. The liquid dispenser as claimed in claim 5, further comprising at least one counterforce surface for manual support of the user's fingers, the at least one counterforce surface being provided in a stationary manner with respect to the nasal applicator.

17. The liquid dispenser as claimed in claim 6, wherein the actuating handle is pivotably movable.

18. The liquid dispenser as claimed in claim 10, wherein each nozzle opening has a minimum clear cross-sectional surface area between 10 μm² and 15 μm².

19. The liquid dispenser as claimed in claim 13, wherein the nozzle plate is manufactured from plastic, and the nozzle openings are formed by injection molding.

20. The liquid dispenser as claimed in claim 14, wherein the nozzle plate has a convexly arched shape.
